(19) United States
(12) Patent Application Publication
(10) Pub. No.: US 2015/0051579 A1
(43) Pub. Date: Feb. 19, 2015

(54) DEVICES HAVING THERMOCHROMIC RESPONSE INDICATORS

(71) Applicant: MEDIMMUNE, LLC, Gaithersburg, MD (US)

(72) Inventors: Jonathan Chung, Gaithersburg, MD (US); Benjamin Bernstein, Gaithersburg, MD (US); Paul Roussel, Gaithersburg, MD (US)

(21) Appl. No.: 14/390,275

(22) PCT Filed: Mar. 13, 2013
(86) PCT No.: PCT/US13/30887

§ 371 Date: Oct. 2, 2014

Related U.S. Application Data
(60) Provisional application No. 61/619,748, filed on Apr. 3, 2012.

(51) Int. Cl. A61M 5/31; A61M 5/20; A61M 5/30

(52) U.S. Cl. CPC A61M 5/3129; A61M 5/30; A61M 5/20; A61M 2205/59
USPC 604/506; 604/189; 604/68

(57) ABSTRACT

The present invention relates to medical devices, in particular autoinjectors and syringes having one or more thermochromic response indicators that are sensitive to temperature changes of a drug formulation or solution. The present invention provides for a pre-filled delivery device comprising a syringe, a cartridge, or a capsule having a barrel, where the barrel is marked on the outer surface with a first thermochromic indicator, and wherein the thermochromic indicator is responsive to temperature changes of the drug formulation or solution within the barrel.

### FIELD

The technology relates to pre-filled delivery devices having one or more thermochromic response indicators that are sensitive to temperature changes of a drug formulation or solution contained within the pre-filled delivery device.

### BACKGROUND

Pre-filled delivery devices are often used for administration of drug formulation or solution to patients. In patients with chronic illnesses, this drug formulation or solution is often self-administered. While pre-filled delivery devices have provided a means for treatment of chronic illnesses, components of pre-filled delivery devices have been susceptible to certain design flaws. These design flaws have presented risks to patients, such as inadvertent injury or chronic under-dosing.

The cost of improper delivery can be high. Patients suffer for not being effectively treated and there is a loss of drug and resources. Certain pre-filled delivery devices, such as the Enbrel SureClick® autoinjectors, have been the subject of a recall for having malfunctioning components. This recall resulted in a loss of approximately 2.95 million doses at a cost of about 3.54 billion dollars.

Numerous adjustments have been made to standard pre-filled delivery devices to ensure, for example, that patients properly remove a rubber shield that protects the needle prior to self-administration, or that patients are able to view the volumetric level of the drug formulation inside the primary package to confirm that the correct volume is present. However, these adjustments do not account for one critical issue for drug to be appropriately administered, that of the temperature of the drug formulation prior to administration.

Typically, in settings of chronic disease conditions where drug is self-administered, the drug formulation or solution is stored at a cold temperature (e.g., in a refrigerator), and then taken out of the refrigerator and allowed to come to room temperature prior to administration.

If the drug formulation or solution is administered prior to attaining a recommended target temperature, administration can be painful for patients. When administration is painful, patients often will not complete administration of the full dose, which can result in ineffective treatment. Over time, pain associated with self-administration can lead to a decrease in patient compliance.

In addition, if the drug formulation or solution does not rise to the appropriate target temperature, the active component of the drug may not fully dissolve in solution. It may, for example, aggregate and stick to the inside surface of the primary package, or aggregate such that it cannot be delivered through the needle or orifice without shearing, or causing damage to the needle or orifice itself. Drug formulation or solution that is too cold also typically exhibits higher viscosity, making it more difficult and painful to administer, particularly for those patients who have limited or diminished dexterity due to their disease or illness. Additionally, some delivery devices may prove unable to administer a complete dose when the drug formulation or solution is too viscous (due to low temperature at the time of operation).

While improvements in some design aspects of pre-filled devices may allow for better barrel or needle structures, these improvements do not ensure that what is being administered is at the correct temperature.

Thus, there is a need in the art to provide for pre-filled delivery devices that are designed such that patients can know that the drug formulation or solution that is being administered or self-administered attains an appropriate target temperature prior to administration.

The present application addresses this problem by providing pre-filled delivery devices that have one or more thermochromic response indicators positioned and applied on the pre-filled delivery devices such that patients can more accurately self-administer drugs for chronic conditions.

### SUMMARY OF THE INVENTION

The present invention provides for a pre-filled delivery device comprising a syringe, a cartridge, or a capsule having a barrel, where the barrel is marked on the outer surface with a first thermochromic indicator, and wherein the thermochromic indicator is responsive to temperature changes of the drug formulation or solution within the barrel.

In certain embodiments, the first thermochromic indicator is a first color when the drug formulation or solution is at a temperature of between 2-8° C. or between 4-5° C. and a second color when the drug formulation or solution is at a temperature of between 15-25%, 16-25%, 17-25%, 18-25%, 19-25%, 20-25° C. or between 22-25° C. In further embodiments, the surface of the pre-filled delivery device that houses the primary package is marked on the outer surface with a second thermochromic indicator.

In certain embodiments, the second thermochromic indicator is a first color when the drug formulation or solution is at a temperature of less than about 40° C. and a second color when the drug formulation or solution is at a temperature of less than about 40° C.

In certain embodiments, the pre-filled delivery device contains a drug formulation or solution for the treatment of a chronic condition. A chronic condition to be treated by use of the present invention can be rheumatoid arthritis (RA), osteoarthritis (OA), chronic back pain, systemic lupus erythematosus (SLE), and multiple sclerosis (MS), asthma, chronic obstructive pulmonary disease (COPD), and pulmonary fibrosis.

In certain other embodiments, the chronic condition is treated with a drug formulation comprising an antibody or fragment thereof that specifically binds to tumor necrosis factor (TNF), nerve growth factor (NGF), interferon alpha (IFNα), or interferon alpha receptor (IFNαR), IL-13, GM-CSFRα, IgE or IL5R. In further embodiments, the antibody or fragment thereof comprises one or more CDRs as disclosed in Table 1 or comprises the VH and/or VL of an antibody as disclosed in Table 2.

In particular embodiments, the pre-filled delivery device is selected from the group consisting of a pre-filled syringe, an autoinjector comprising a pre-filled syringe, a pre-filled cartridge, an autoinjector comprising a pre-filled cartridge, and a pre-filled needle-free injection device.

The invention further provides for a kit or package comprising one or more of the pre-filled delivery devices of the present invention. In further embodiments, the kit or package comprises instructions describing how the color(s) of each of the thermochromic response indicators correspond to temperature of the drug formulation or solution.

The invention further provides for a method of treating a chronic condition using the pre-filled delivery device of the invention. In particular embodiments, the chronic condition can be rheumatoid arthritis, osteoarthritis, chronic back pain, systemic lupus erythematosus (SLE), and multiple sclerosis, asthma, chronic obstructive pulmonary disease, or pulmonary fibrosis.

### BRIEF DESCRIPTION OF THE DRAWINGS

The drawings illustrate embodiments herein and are not limiting. For clarity and ease of illustration, the drawings are not made to scale and, in some instances, various aspects may be shown exaggerated or enlarged to facilitate an understanding of particular embodiments.

FIGS. 1 is a diagram of a syringe placed within an autoinjector marked with a first thermochromic indicator and a second thermochromic indicator.

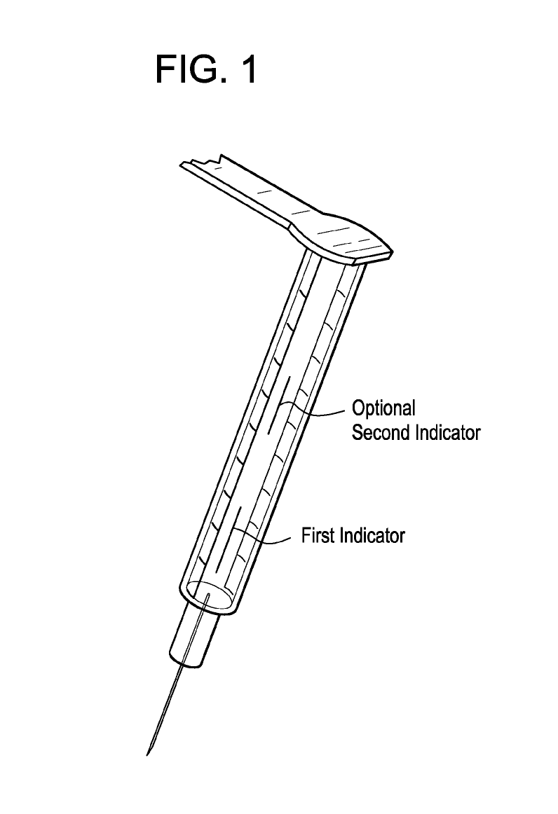

FIGS. 2 is a diagram of an autoinjector having a syringe placed within its barrel, where the barrel of the syringe is marked with a first and second thermochromic indicator.

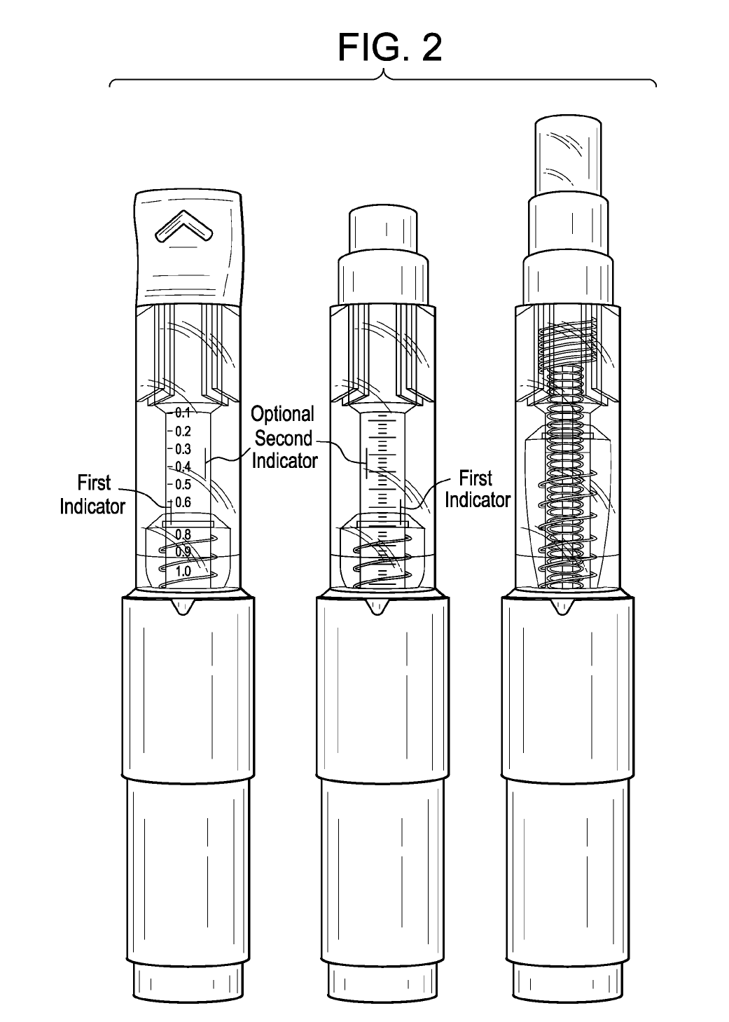

### DETAILED DESCRIPTION OF THE INVENTION

Pre-Filled Delivery Devices

A pre-filled delivery device corresponds to a pre-filled syringe, an autoinjector containing a pre-filled syringe, a pre-filled cartridge, a pre-filled needle-free injection device, a pre-filled vial, or equivalents thereof.

In certain embodiments, the pre-filled delivery device is marked with a first thermochromic indicator, where the first thermochromic indicator is responsive to temperature changes of the drug formulation or solution. The first thermochromic indicator can be marked on the outer surface of the barrel of the syringe, cartridge or needle-free injection device. In the case of an autoinjector, the first thermochromic indicator can be marked on the barrel of the pre-filled syringe that is contained within the autoinjector.

In certain other embodiments, the first thermochromic indicator is utilized to determine whether a drug formulation or solution has attained an appropriate target temperature for administration, such as room temperature, subsequent to the pre-filled delivery device being removed from storage, such as a refrigerator or cold storage container. In particular embodiments, the first thermochromic indicator is of a first color when the drug solution or formulation has a temperature in the range of 1-8° C., 2-8° C., 3-5° C., 4-7° C., or has a temperature at or about 2, 3, 4, 5, 6, 7 or 8° C. In additional embodiments, the first thermochromic indicator is of a second color when the drug solution or formulation has a temperature in the range of 15-25%,16-25%, 17-25%, 18-25%, 19-25%, 20-25° C., or has a temperature at or about 15, 16, 17, 18, 19, 20, 21, 22, 23, 24 or 25° C. A range of 20-25° C. or a temperature at or about 20, 21, 22, 23, 24 or 25° C. is a temperature that corresponds to room temperature.

In certain embodiments, the first color of a thermochromic indicator can correspond to any one of a number of standard printing colors, where the thermochromic ink generating the color is commercially available or known to one of ordinary skill in the art. The first color can correspond to a color selected from the group consisting of red, green, blue, yellow, purple, orange, brown, black, white, or can correspond to a color that is a variation or shade of the colors above, including primrose yellow, lemon yellow, medium yellow, fire red, brilliant orange, ultra blue, warm red, opaque black, reflex blue, opaque white, high intensity white, carmine, magenta, violet, rodamine red, tinting white, tinting black. The second color can correspond to any one of the colors indicated above, and preferably corresponds to a color that is different or contrasting to the first color.

For example, in an instance where the first color of a first thermochromic indicator is red, or a variation thereof, the second color of a first thermochromic indicator is green or blue, or a variation thereof. In an instance where the first color of a first thermochromic indicator is blue, or a variation thereof, the second color of a first thermochromic indicator can be red or orange, or a variation thereof. In other instances, the first color of a first thermochromic indicator is orange or a variation thereof, and the second color of a first thermochromic indicator is purple or blue or a variation thereof. Exemplary thermochromic ink that can be utilized for the thermochromic indicators of the present invention is available, for example, from Hallcrest and other companies whose technology relates to color changing graphic technology.

With the application of the first thermochromic indicator, the change from the first color to the second color will be an indication that the drug formulation or solution has attained the appropriate target temperature. Thus, the patient will know not to administer the drug formulation or solution until the first thermochromic indicator displays the second color.

In other embodiments, the pre-filled delivery device is marked with a second thermochromic indicator, where the second thermochromic indicator is also responsive to temperature changes of the drug formulation or solution. The second thermochromic indicator is utilized to determine whether a drug formulation or solution has warmed to a temperature that has exceeded the appropriate target temperature, and thus may not be active or efficacious. In particular embodiments, the second thermochromic indicator is of a first color when the drug solution or formulation has a temperature in the range of less than about 40° C. and is of a second color when the drug solution or formulation has a temperature in the range of greater than about 40° C.

The second thermochromic indicator can be marked on the outer surface of the barrel of the syringe, cartridge or needle-free injection device, or, in the case of an autoinjector, on the barrel of the pre-filled syringe that is contained within the autoinjector. The second thermochromic indicator is preferably marked in a different location to the first thermochromic indicator, for example, on the opposite side of the barrel of the first thermochromic indicator. Alternatively, for example, if the first thermochromic indicator is applied closer towards the needle of the syringe or the tip of the delivery device, the second thermochromic indicator can be applied closer to the plunger on the opposite end of the barrel has the needle or the tip of the delivery device.

A pre-filled delivery device of the present invention can be used to administer a single dose or multiple doses. Such pre-filled delivery devices can be singly packaged or packaged together with additional pre-filled delivery devices. A package comprising a pre-filled delivery device can include instructions for the patient describing the corresponding temperature(s) and color(s) of each of the thermochromic response indicators.

Syringes

A pre-filled syringe of the present invention can be any one of a number of commercially available pre-filled syringes, or pre-filled syringes having a design known to one of ordinary skill in the art, having one or more additional features. Such features can include, for example, a retractable needle, an extendable needle having a guard device or shield, or a pre-filled syringe having a barrel with volume gradation marks or other indicators. Example of syringes that can be used include, for example, those described in U.S. Pat. Nos. 6,086,566; 6,428,519; 6,565,540; 7,935,087; 7,041,087; 6,977,901; 7,141,286; 6,544,235; 7,699,811; the disclosure of each which are herein incorporated by reference in their entirety. A volume of drug substance or formulation can correspond to 0.3 ml, 0.5 ml, 1 ml, 2 ml, 3 ml, 4 ml, 5 ml, 6 ml, 7 ml, 8 ml, 9 ml, 10 ml, 15 ml, 20 ml, 25 ml, 30 ml, 35 ml, 40 ml, 45 ml, 50 ml, 60 ml, 70 ml, 80 ml, 90 ml, 100 ml, 200 ml, 300 ml, 400 ml, or 500 ml.

Autoinjectors

An autoinjector of the present invention can be any one of a number of commercially available autoinjectors, or autoinjectors having a design known to one of ordinary skill in the art, having one or more additional features. An autoinjector, sometimes referred to as a pen, is an automatic injection device designed to facilitate delivery of a dose of medicament to a patient through a hypodermic needle, the injection usually being administered by the patient themselves.

Autoinjectors are often designed to be used in conjunction with a standard drug presentation, for example, a pre-filled syringe as described above. e.g. a syringe comprising a needle, where the barrel of the syringe is prefilled with a drug formulation or solution. Exemplary autoinjectors available today include HUMIRA® Pen and Enbrel SureClick®.

An autoinjector works by delivering an injection automatically upon actuation by the patient pressing a button, moving a lever or part of a housing. Autoinjectors are designed to facilitate injection procedures over those required by manual use of common syringes and to secure a proper injection result highly independent of operational circumstances. Autoinjectors are typically used in non-hospital environments, sometimes in emergency situations, and by non-professionals like unskilled assistants or the patients themselves, which operator groups may include sick, disabled, elderly and child persons. The autoinjectors provide at least an automatic injection step in which stored energy, for example from a compressed spring, is released by a trigger to act on a syringe piston or plunger for expulsion of syringe content.

Frequently the autoinjectors also provide an automatic penetration step in which stored energy is used for propulsion of the syringe from a rear position, in which the needle is hidden, to a front position, in which the needle is at least partially exposed, to thereby relieve the patient from the, sometimes fearful, task of inserting the needle through the skin and to secure an always appropriate penetration depth once the autoinjector front has been placed against the skin. Autopenetration and autoinjection may take place concurrently, e.g. in simple devices or for the intentional purpose of allowing for an over depth distributed injection. Normally it is desirable to limit injection until the needle has reached or is close to its target location. Some injectors achieve this by utilizing a single force system, while others apply single or dual drive systems.

Autoinjectors may also provide an automatic needle retraction step in which stored energy, typically stored during the penetration movement in a weaker return counterspring, acts to push the syringe back into the autoinjector after completed injection in order to relieve the user from the task and risk of withdrawal, to verify sequence completion to the user and to prevent inadvertent needle pricks after use. Again, this function may need a control mechanism enabling action of the return spring only after completed injection, normally accomplished by separation of the penetration and injection forces from the syringe at a certain forward extreme for the piston or plunger, freeing the return spring for action.

Examples of autoinjectors are disclosed, for example, in U.S. Pat. Nos. 7,811,254; 7,381,201; 6,371,939; 6,270,479; U.S. Publ. Nos. 20110313364; 20110282278 20100152655; 20100130930; 20100016795, the disclosure of each which is herein incorporated by reference in its entirety.

Cartridges

A cartridge of the present invention can be any one of a number of commercially available cartridges, or cartridges having a design known to one of ordinary skill in the art, having one or more additional features. Cartridges can be pre-filled with a single dose of drug, or can be pre-filled with multiple doses. Some cartridges may contain multiple pre-filled chambers to facilitate reconstitution of lyophilized (freeze-dried) drug products.

Needle-Free Injection Devices

A needle-free injection device of the present invention can be any one of a number of commercially available needle-free injection devices, or needle-free injection devices having a design known to one of ordinary skill in the art, having one or more additional features. Needle-free injection devices can use pressurized gas to power a hypodermic jet injection or use other high-pressure fluids that drive the piston and deliver one or more injections. In certain examples, delivery is achieved through a spring-powered device.

An example of a needle-free injection device is disclosed, for example, in U.S. Pat. No. 6,641,554, the disclosure of each which is herein incorporated by reference in its entirety.

Disease Indications and Drug Formulations

The present invention provides for easier, safer, and more dose-appropriate administration of drug formulations and products for the long-term treatment of chronic diseases, including, but not limited to rheumatoid arthritis (RA), osteoarthritis (OA), chronic back pain, systemic lupus erythematosus (SLE), multiple sclerosis (MS), asthma, chronic obstructive pulmonary disease (COPD), and pulmonary fibrosis.

Drug formulations or solutions can be standard lyophilized or liquid formulations known to one of ordinary skill in the art. A pre-filled delivery device of the present invention can be utilized to administer or self-administer such drug formulations or solutions. Administration or self-administration can be performed, for example, by methods known in the art, and in particular, intramuscularly, intradermally, or subcutaneously.

A drug formulation or solution of the invention can comprise an antibody or a fragment thereof. In certain embodiments, the antibody or fragment thereof specifically binds to the alpha chain of the receptor for granulocyte macrophage colony stimulating factor (GM-CSFRα). Exemplary anti-GM-CSFRα antibodies are disclosed, for example, in U.S. Publ. No. 2009/0130093, the entire contents of which are herein incorporated by reference. In further embodiments, the antibody or fragment thereof that specifically binds to GM-CSFRα corresponds to the antibody designated as Antibody 6 in U.S. Publ. No. 2009/0130093, or an antibody or fragment thereof comprising the CDRs of the antibody designated as Antibody 6.

In certain embodiments, the antibody or fragment thereof specifically binds to human IL-13 and neutralize IL-13 activity. Exemplary IL-13 antibodies are disclosed, for example, in U.S. Pat. No. 7,829,090, the contents of which are herein incorporate by reference in their entirety.

In other embodiments, the antibody or fragment thereof specifically binds to a human interferon alpha polypeptide, disclosed, for example, in U.S. Pat. No. 7,741,449, the contents of which are herein incorporated by reference in their entirety. In particular embodiments, the antibody or fragment thereof that specifically binds to human interferon alpha polypeptide corresponds to the antibody designated as 13H5 in U.S. Pat. No. 7,741,449, or an antibody or fragment thereof comprising the CDRs of the antibody designated as 13H5. In certain other embodiments, the antibody or fragment thereof binds to human interferon alpha receptor polypeptide, examples of which are disclosed in US Publ. No. 2011/0059078, the disclosure of which is herein incorporated by reference.

In other embodiments, the antibody or fragment thereof specifically binds to a human interleukin-5 receptor alpha chain (IL-5Rα) polypeptide, disclosed, for example, in U.S. Pat. No. 7,238,354, the contents of which are herein incorporated by reference in their entirety.

In further embodiments, the drug formulation or solution is any one of the formulations and solutions, disclosed, for example in US Publ. No. 2011/0086038. Such formulations are used for the treatment of IL-13 related disorders, including asthma, atopic dermatitis, allergic rhinitis, fibrosis, inflammatory bowel disease and Hodgkin's lymphoma.

In other embodiments, the antibody or fragment thereof specifically binds to a human interferon alpha receptor (INFαR) polypeptide, disclosed, for example, in U.S. Pat. No. 7,662,381, the contents of which are herein incorporated by reference in their entirety.

In other embodiments, the antibody or fragment thereof specifically binds to a human IgE polypeptide, disclosed, for example, in U.S. Pat. Nos. 7,959,917 and 8,389,704, the contents of which are herein incorporated by reference in their entirety.

In certain embodiments, the drug formulation or solution of the present invention is a high concentration liquid formulation comprising an antibody or fragment thereof that specifically bind to a human interferon alpha polypeptide, which formulations exhibit stability, low to undetectable levels of antibody fragmentation, low to undetectable levels of aggregation, and very little to no loss of the biological activities of the antibodies, even during long periods of storage.

Such high concentration liquid formulations can be administered for preventing, treating, managing or ameliorating symptoms associated with an interferon alpha mediated disease or disorder (for example, but not limited to, systemic lupus erythematosus, multiple sclerosis, inflammatory bowel disease, insulin dependent diabetes mellitus, psoriasis, autoimmune thyroiditis, rheumatoid arthritis and glomerulonephritis, transplant rejection, graft versus host disease). Such formulations are disclosed, for example, in U.S. Publ. No. US 2010/0209434, the contents of which are herein incorporated by reference in their entirety.

Table 1 below lists VH and VL CDR sequences of
exemplary antibodies of the invention:

| | VH | VH | VH | VL | VL | VL |
|---|---|---|---|---|---|---|
| Ab | CDR1 | CDR2 | CDR3 | CDR1 | CDR2 | CDR3 |
| Anti- | NYGLS | WISANNG | DSSSNW | GGNNIGSI | DDGDRPS | QVWDT |
| IL13 | (SEQ ID | DTNYGQE | ARWFFDL | CLVH | (SEQ ID | GSDPVV |
| | NO: 1) | FQG (SEQ | (SEQ ID | (SEQ ID | NO: 5) | (SEQ ID |
| | | ID NO: 2) | NO: 3) | NO: 4) | | NO: 6) |
| Anti- | ELSIH | GFDPEENEI | VGSFSPL | TGSGSNI | HNNKRPS | ATVEAG |
| GM- | (SEQ ID | VYAQRFQG | TLGL | GAPYDVS | (SEQ ID | LSGSV |
| CSFRα | NO: 7) | (SEQ ID | (SEQ ID | (SEQ ID | NO: 11) | (SEQ ID |
| | | NO: 8) | NO: 9) | NO: 10) | | NO: 12) |
| Anti- | SYSIS | WISVYNGNT | DPIAAGY | RASQSVS | GASSRAT | QQYGS |
| IFNa | (SEQ ID | NYAQKFQG | (SEQ ID | STYLA | (SEQ ID | SPRT |
| | NO: 13) | (SEQ ID | NO: 15) | (SEQ ID | NO: 17) | (SEQ ID |
| | | NO: 14) | | NO: 16) | | NO: 18) |
| Anti-IL- | DYGMA | AISSGGSYI | RGFYGN | RANESVDH | AASNQGS | QQSKDV |
| 5Rα | (SEQ ID | HFPDSLKG | YRAMDY | NGVNFMN | (SEQ ID | PWT |
| | NO: 19) | (SEQ ID | (SEQ ID | (SEQ ID | NO: 23) | (SEQ ID |
| | | NO: 20) | NO: 21) | NO: 22) | | NO: 24) |
| Anti-IL- | SYVIH | YINPYNDGT | EGIRYYG | GYSEDIINYLN | HTSRLQS | QQGYTL |
| 5Rα | (SEQ ID | KYNERFKG | LLGDY | (SEQ ID | (SEQ ID | PYT |
| | NO: 25) | (SEQ ID | (SEQ ID | NO: 28) | NO: 29) | (SEQ ID |
| | | NO: 26) | NO: 27) | | | NO: 30) |
| | DTYMH | RIDPANGNT | GLRLRFFDY | SASSSVSYMH | DTSKLAS | QQWSS |
| | (SEQ ID | KSDPKFQA | (SEQ ID | (SEQ ID | (SEQ ID | NPPIT |
| | NO: 31) | (SEQ ID | NO: 33) | NO: 34) | NO: 35) | (SEQ ID |
| | | NO: 32) | | | | NO: 36) |
| Anti- | NYWIA | IIYPGDSDI | HDIEGFDY | RASQSVS | GASSRAT | QQYDS |
| IFNaR | (SEQ ID | RYSPSFQ | (SEQ ID | SSFFA | (SEQ ID | SAIT |
| | NO: 47) | (SEQ ID | NO: 49) | (SEQ ID | NO: 51) | (SEQ ID |
| | | NO: 48) | | NO: 50) | | NO: 52) |

Table 2 below lists VH and VL sequences of
exemplary antibodies of the invention:

| Ab | VH | VL |
|---|---|---|
| Anti- | QVQLVQSGAEVKKPGASV | SYVLTQPPSVSVAPGKTARITC |
| IL13 | KVSCKASGYTFTNYGLSW | GGNIIGSKLVHWYQQKPGQAPV |
| | VRQAPGQGLEWMGWISAN | LVIYDDGDRPSGIPERFSGSNS |
| | NGDTNYGQEFQGRVTMTT | GNTATLTISRVEAGDEADYYCQ |
| | DTSTSTAYMELRSLRSD | VWDTGSDPVVFG GGTKLTVL |
| | DTAVYYCARDSSSSWAR | (SEQ ID NO: 38) |
| | WFFDLWGRGTLVTVSS | |
| | (SEQ ID NO: 37) | |
| Anti- | QVQLVQSGAEVKKPGASVKVS | QSVLTQPPSVSGAPGQRVTI |
| GM- | CKVSGYTLTELSIHWVRQ | SCTGSGSNIGAPYDVSWYQQ |
| CSFRα | APGKGLEWMGGFDPEENEI | LPGTAPKLLIYHNNKRPSGVP |
| | VYAQRFQGRVTMTEDTSTD | DRFSGSKSGTSASLAITGLQ |
| | TAYMELSSLRSEDTAVYYCA | AEDEADYYCATVEAGLSG |
| | IVGSFSPLTLGLWGQGTMV | SVFGGGTKLTVLGA |
| | TVSS (SEQ ID NO: 39) | (SEQ ID NO: 40) |
| Anti- | QVQLVQSGAEVKKPGASVKV | EIVLTQSPGTLSLSPGERATL |
| IFNa | SCKASGYTFTSYSISWVRQA | SCRASQSVSSTYLAWYQQKP |
| | PGQGLEWMGWISVYNGNTN | GQAPRLLIYGASSRATGIPDR |
| | YAQKFQGRVTMTTDTSTST | FSGSGSGTDFTLTISRLEPED |
| | AYLELRSLRSDDTAVYYCA | FAVYYCQQYGSSPRTFGQGTK |
| | RDPIAAGYWGQGTLVTVSS | VEIK |
| | (SEQ ID NO: 41) | (SEQ ID NO: 42) |
| Anti- | EVQLVQSGAEVKKPGESLKI | EIVLTQSPGTLSLSPGERATLS |
| IFNaR | SCKGSGYIFTNYWIAWVRQM | CRASQSVSSSFFAWYQQKPGQA |
| | PGKGLESMGIIYPGDSDIRY | PRLLIYGASSRATGIPDRLSGS |
| | SPSFQGQVTISADKSITTAY | GSGTDFTLTITRLEPEDFAVYY |
| | LQWSSLKASDTAMYYCARHD | CQQYDSSAITFGQGTRLEIK |
| | IEGFDYWGRGTLVTVSS | (SEQ ID NO: 44) |
| | (SEQ ID NO: 43) | |
| Anti- | EVQLVQSGAEVKKPGATVKIS | QSVLTQPPSVSGAPGQRVTI |
| IgE | CKVYGYIFTDYNIYWVRQAPG | SCTGSSSNIGAGYDVHVVYQ |
| | KGLEWMGLIDPDNGETFYAEK | QLPGTAPKLLIYDNFNRPSG |
| | FQGRATMTADTSSDRAYMELS | VPDRFSGSKSGTSASLAITG |
| | SLRFEDTAVYYCATVMGKWIK | LQAEDEADYYCQSYDSPTLT |
| | GGYDYWGRGTLVTVSS | SPFGTGTKLTVLG |
| | (SEQ ID NO: 45) | (SEQ ID NO: 46) |

### EXAMPLE

### Thermochromic Indicator for Use on Pre-Filled Syringe (PFS)

The length of time it would take for 1.0 mL with 100 mg/mL of an anti-IL-5Rα drug substance, stored in a 1 mL long pre-filled syringe (PFS) and assembled in a BD Physioject Autoinjector (AI), to reach a room temperature of 18° C. was experimentally determined. The results confirmed that the AI shell equilibrates to room temperature more quickly than the drug substance, highlighting the need for a means of determining the drug substance temperature.

The samples were assembled in a 2-8° C. cold room storage by the following steps:
1. Drilled 1 hole roughly 1.5 cm from the bottom edge into the top housing of the BD Physioject.
2. Threaded thermocouple through hole.
3. Removed the stopper from the PFS using a threaded plunger rod.
4. The stopper and thermocouple were coupled together to bypass the ribs during the insertion of the stopper and allow air pressure to escape when replacing the stopper back into the PFS.
5. Placed stopper just touching the DS in the syringe without allowing the fluid to bypass stopper ribs.
6. Removed plunger rod.
7. Inserted syringe into bottom housing of the autoinjector.
8. Snapped together the top and bottom housings together, while removing as much thermocouple slack as possible.

The second thermocouple was placed on the outer housing of the AI and securely adhered to the surface using masking tape.

The samples were transferred from cold room storage in Styrofoam containers with cold and frozen packs. Once the thermocouples were connected to the thermocouple data loggers, they were removed from the Styrofoam containers and allowed to reach a room temperature of 18 C. The data loggers recorded the time that it took for both the drug substance and the shell to warm to room temperature.

As seen in Table 3 below, the average time for the drug substance to reach 18° C. was approximately 29 minutes, with a range of 24 to 40 minutes amongst the three samples. In contrast, the time it took for the housing to reach 18° C. was approximately 16 minutes, with a range of 12 to 25 minutes amongst the three samples. On average, it took approximately a full 13 minutes longer on average for the drug substance to reach room temperature in comparison with the AI housing.

TABLE 3
Table 1 - Minutes for Samples to Equilibrate
Time Table (min)

| | Drug Substance | AI Housing |
|---|---|---|
| Fill/Sample | 18° C. | 18° C. |
| 1mL_1 | 24.5 | 12.0 |
| 1mL_2 | 39.5 | 25.0 |
| 1mL_3 | 24.0 | 12.0 |
| Average Time (mins) | 29.3 | 16.3 |

From the data of Table 3 above, there is a major difference between the time it takes for a drug substance to reach room temperature and the time it takes for the AI housing to reach room temperature. As such, if a temperature indicator was used on the outside of the AI housing, alerting the end-user that the drug substance was at an appropriate temperature to dose, this would have potentially detrimental consequences. With the use of the autoinjector, drug substance injected at a lower temperature in an AI could result in an incomplete dosing, glass PFS breakage, and overall malfunctioning of the AI that could lead to costly recalls. Therefore, this data supports the fact that a thermal indicator directly printed or adhered to the outer surface of a PFS would be one means of accurately determining drug temperature. As shown above, when the thermal indicator was placed onto the surface of the PFS rather than the AI, this allowed for an accurate indication of the temperature of the drug substance.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

What is claimed is:

1. A pre-filled delivery device comprising a syringe, a cartridge, or a capsule having a barrel, where the barrel is marked on the outer surface with a first thermochromic indicator, and wherein the thermochromic indicator is responsive to temperature changes of a drug formulation or solution within the barrel.

2. The pre-filled delivery device of claim 1, wherein the first thermochromic indicator is a first color when the drug formulation or solution is at a temperature of between 2-8° C. and a second color when the drug formulation or solution is at a temperature of between 18-25° C.

3. The pre-filled delivery device of claim 1 or 2, wherein the first thermochromic indicator is a first color when the drug formulation or solution is at a temperature of between 2-8° C. and a second color when the drug formulation or solution is at a temperature of between 20-25° C.

4. The pre-filled delivery device of any of claims 1-3, wherein the first thermochromic indicator is a first color when the drug formulation or solution is at a temperature of between 4-5° C., a second color when the drug formulation or solution is at a temperature of between 22-25° C.

5. The pre-filled delivery device of any of claim 1-4, wherein the surface of the pre-filled delivery device is marked on the outer surface with a second thermochromic indicator.

6. The pre-filled delivery device of claim 5, wherein the second thermochromic indicator is a first color when the drug formulation or solution is at a temperature of less than about 40° C. and a second color when the drug formulation or solution is at a temperature of less than about 40° C.

7. The pre-filled delivery device of any of claims 1-6, wherein the drug formulation or solution is for the treatment of a chronic condition.

8. The pre-filled delivery device of claim 7, wherein the chronic condition is selected from the group consisting of rheumatoid arthritis (RA), osteoarthritis (OA), chronic back pain, systemic lupus erythematosus (SLE), and multiple sclerosis (MS).

9. The pre-filled delivery device of claim 7, wherein the chronic condition is selected from the group consisting of asthma, chronic obstructive pulmonary disease (COPD), and pulmonary fibrosis.

10. The pre-filled delivery device of claim 8, wherein the chronic condition is treated with a drug formulation comprising an antibody or fragment thereof that specifically binds to IgE, tumor necrosis factor (TNF), nerve growth factor (NGF), interferon alpha (IFNα), or interferon alpha receptor (IFNαR).

11. The pre-filled delivery device of claim 9, wherein the chronic condition is treated with a drug formulation comprising an antibody or fragment thereof that specifically binds to IgE, IL-13, GM-CSFRα or IL5R.

12. The pre-filled delivery device of claim 11, wherein the antibody or fragment thereof that specifically binds to IL-13 comprises one or more CDRs of the anti-IL13 antibody as disclosed in Table 1.

13. The pre-filled delivery device of claim 12, wherein the antibody or fragment thereof that specifically binds to IL-13 comprises the VH and/or VL of the anti-IL13 antibody as disclosed in Table 2.

14. The pre-filled delivery device of claim 11, wherein the antibody or fragment thereof that specifically binds to GM-CSF comprises one or more CDRs of an anti-GM-CSFRα antibody as disclosed in Table 1.

15. The pre-filled delivery device of claim 14, wherein the antibody or fragment thereof that specifically binds to GM-CSF comprises the VH and/or VL of the anti-GM-CSFRα antibody as disclosed in Table 2.

16. The pre-filled delivery device of claim 10, wherein the antibody or fragment thereof that specifically binds to IFNα comprises one or more CDRs of an anti-IFNα antibody as disclosed in Table 1.

17. The pre-filled delivery device of claim 16, wherein the antibody or fragment thereof that specifically binds to IFNα comprises the VH and/or VL of the anti-IFNα antibody as disclosed in Table 2.

18. The pre-filled delivery device of claim 10, wherein the antibody or fragment thereof that specifically binds to IFNαR comprises one or more CDRs of an anti-IFNα antibody as disclosed in Table 1.

19. The pre-filled delivery device of claim 18, wherein the antibody or fragment thereof that specifically binds to IFNαR comprises the VH and/or VL of the anti-IFNα antibody as disclosed in Table 2.

20. The pre-filled delivery device of any one of claims 1-19, wherein the pre-filled delivery device is selected from the group consisting of a pre-filled syringe, an autoinjector comprising a pre-filled syringe, a pre-filled cartridge, an autoinjector comprising a pre-filled cartridge, and a pre-filled needle-free injection device.

21. The pre-filled delivery device of any one of claims 1-20, wherein the pre-filled delivery device is a pre-filled syringe.

22. The pre-filled delivery device of any one of claims 1-20, wherein the pre-filled delivery device is an autoinjector comprising a pre-filled syringe.

23. A kit or package comprising one or more of the pre-filled delivery device according to any one of claims 1-22.

24. The kit or package of claim 23, further comprising instructions describing how the color(s) of each of the thermochromic response indicators correspond to temperature of the drug formulation or solution.

25. A method of treating a chronic condition using the pre-filled delivery device of any one of claims 1-22.

26. A method of claim 25, wherein the chronic condition is selected from the group consisting of rheumatoid arthritis, osteoarthritis, chronic back pain, systemic lupus erythematosus (SLE), and multiple sclerosis.

27. A method of claim 25, wherein the chronic condition is selected from the group consisting of asthma, chronic obstructive pulmonary disease, and pulmonary fibrosis.


SEQUENCE LISTING (52 sequences)

SEQ ID NO: 1 - protein, 5 aa, Artificial Sequence
  Description of Artificial Sequence Synthetic peptide
NYGLS

SEQ ID NO: 2 - protein, 17 aa, Artificial Sequence
  Description of Artificial Sequence Synthetic peptide
WISANNGDTNYGQEFQG

SEQ ID NO: 3 - protein, 13 aa, Artificial Sequence
  Description of Artificial Sequence Synthetic peptide
DSSSNWARWFFDL

SEQ ID NO: 4 - protein, 12 aa, Artificial Sequence
  Description of Artificial Sequence Synthetic peptide
GGNNIGSICLVH

SEQ ID NO: 5 - protein, 7 aa, Artificial Sequence
  Description of Artificial Sequence Synthetic peptide
DDGDRPS

SEQ ID NO: 6 - protein, 11 aa, Artificial Sequence
  Description of Artificial Sequence Synthetic peptide
QVWDTGSDPVV

SEQ ID NO: 7 - protein, 5 aa, Artificial Sequence
  Description of Artificial Sequence Synthetic peptide
ELSIH

SEQ ID NO: 8 - protein, 17 aa, Artificial Sequence
  Description of Artificial Sequence Synthetic peptide
GFDPEENEIVYAQRFQG

SEQ ID NO: 9 - protein, 11 aa, Artificial Sequence
  Description of Artificial Sequence Synthetic peptide
VGSFSPLTLGL

SEQ ID NO: 10 - protein, 14 aa, Artificial Sequence
  Description of Artificial Sequence Synthetic peptide
TGSGSNIGAPYDVS

SEQ ID NO: 11 - protein, 7 aa, Artificial Sequence
  Description of Artificial Sequence Synthetic peptide
HNNKRPS

SEQ ID NO: 12 - protein, 11 aa, Artificial Sequence
  Description of Artificial Sequence Synthetic peptide
ATVEAGLSGSV

SEQ ID NO: 13 - protein, 5 aa, Artificial Sequence
  Description of Artificial Sequence Synthetic peptide
SYSIS

SEQ ID NO: 14 - protein, 17 aa, Artificial Sequence
  Description of Artificial Sequence Synthetic peptide
WISVYNGNTNYAQKFQG

SEQ ID NO: 15 - protein, 7 aa, Artificial Sequence
  Description of Artificial Sequence Synthetic peptide
DPIAAGY

SEQ ID NO: 16 - protein, 12 aa, Artificial Sequence
  Description of Artificial Sequence Synthetic peptide
RASQSVSSTYLA

SEQ ID NO: 17 - protein, 7 aa, Artificial Sequence
  Description of Artificial Sequence Synthetic peptide
GASSRAT

SEQ ID NO: 18 - protein, 9 aa, Artificial Sequence
  Description of Artificial Sequence Synthetic peptide
QQYGSSPRT

SEQ ID NO: 19 - protein, 5 aa, Artificial Sequence
  Description of Artificial Sequence Synthetic peptide
DYGMA

SEQ ID NO: 20 - protein, 17 aa, Artificial Sequence
  Description of Artificial Sequence Synthetic peptide
AISSGGSYIHFPDSLKG

SEQ ID NO: 21 - protein, 12 aa, Artificial Sequence
  Description of Artificial Sequence Synthetic peptide
RGFYGNYRAMDY

SEQ ID NO: 22 - protein, 15 aa, Artificial Sequence
  Description of Artificial Sequence Synthetic peptide
RANESVDHNGVNFMN

SEQ ID NO: 23 - protein, 7 aa, Artificial Sequence
  Description of Artificial Sequence Synthetic peptide
AASNQGS

SEQ ID NO: 24 - protein, 9 aa, Artificial Sequence
  Description of Artificial Sequence Synthetic peptide
QQSKDVPWT

SEQ ID NO: 25 - protein, 5 aa, Artificial Sequence
  Description of Artificial Sequence Synthetic peptide
SYVIH

SEQ ID NO: 26 - protein, 17 aa, Artificial Sequence
  Description of Artificial Sequence Synthetic peptide
YINPYNDGTKYNERFKG

SEQ ID NO: 27 - protein, 12 aa, Artificial Sequence
  Description of Artificial Sequence Synthetic peptide
EGIRYYGLLGDY

SEQ ID NO: 28 - protein, 11 aa, Artificial Sequence
  Description of Artificial Sequence Synthetic peptide
GYSEDIINYLN

SEQ ID NO: 29 - protein, 7 aa, Artificial Sequence
  Description of Artificial Sequence Synthetic peptide
HTSRLQS

SEQ ID NO: 30 - protein, 9 aa, Artificial Sequence
  Description of Artificial Sequence Synthetic peptide
QQGYTLPYT

SEQ ID NO: 31 - protein, 5 aa, Artificial Sequence
  Description of Artificial Sequence Synthetic peptide
DTYMH

SEQ ID NO: 32 - protein, 17 aa, Artificial Sequence
  Description of Artificial Sequence Synthetic peptide
RIDPANGNTKSDPKFQA

SEQ ID NO: 33 - protein, 9 aa, Artificial Sequence
  Description of Artificial Sequence Synthetic peptide
GLRLRFFDY

SEQ ID NO: 34 - protein, 10 aa, Artificial Sequence
  Description of Artificial Sequence Synthetic peptide
SASSSVSYMH

SEQ ID NO: 35 - protein, 7 aa, Artificial Sequence
  Description of Artificial Sequence Synthetic peptide
DTSKLAS

SEQ ID NO: 36 - protein, 10 aa, Artificial Sequence
  Description of Artificial Sequence Synthetic peptide
QQWSSNPPIT

SEQ ID NO: 37 - protein, 122 aa, Artificial Sequence
  Description of Artificial Sequence Synthetic polypeptide
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 38 - protein, 108 aa, Artificial Sequence
  Description of Artificial Sequence Synthetic polypeptide
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 39 - protein, 120 aa, Artificial Sequence
  Description of Artificial Sequence Synthetic polypeptide
QVQLVQSGAEVKKPGASVKVSCKVSGYTLTELSIHWVRQAPGKGLEWMGGFDPEENEIVYAQRFQGRVTMTEDTSTDTAYMELSSLRSEDTAVYYCAIVGSFSPLTLGLWGQGTMVTVSS

SEQ ID NO: 40 - protein, 113 aa, Artificial Sequence
  Description of Artificial Sequence Synthetic polypeptide
QSVLTQPPSVSGAPGQRVTISCTGSGSNIGAPYDVSWYQQLPGTAPKLLIYHNNKRPSGVPDRFSGSKSGTSASLAITGLQAEDEADYYCATVEAGLSGSVFGGGTKLTVLGA

SEQ ID NO: 41 - protein, 116 aa, Artificial Sequence [sequence omitted]

SEQ ID NO: 42 - protein, 108 aa, Artificial Sequence [sequence omitted]

SEQ ID NO: 43 - protein, 117 aa, Artificial Sequence [sequence omitted]

SEQ ID NO: 44 - protein, 108 aa, Artificial Sequence [sequence omitted]

SEQ ID NO: 45 - protein, 121 aa, Artificial Sequence
  Description of Artificial Sequence Synthetic polypeptide
EVQLVQSGAEVKKPGATVKISCKVYGYIFTDYNIYWVRQAPGKGLEWMGLIDPDNGETFYAEKFQGRATMTADTSSDRAYMELSSLRFEDTAVYYCATVMGKWIKGGYDYWGRGTLVTVSS

SEQ ID NO: 46 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYDNFNRPSGVPDRFSGSKSGTSASLAITGLQAEDEADYYCQSYDSPTLTSPFGTGTKLTVLG

SEQ ID NO: 47 - protein, 5 aa, Artificial Sequence
  Description of Artificial Sequence Synthetic peptide
NYWIA

SEQ ID NO: 48 - protein, 17 aa, Artificial Sequence
  Description of Artificial Sequence Synthetic peptide
IIYPGDSDIRYSPSFQG

SEQ ID NO: 49 - protein, 8 aa, Artificial Sequence
  Description of Artificial Sequence Synthetic peptide
HDIEGFDY

SEQ ID NO: 50 - protein, 12 aa, Artificial Sequence
  Description of Artificial Sequence Synthetic peptide
RASQSVSSSFFA

SEQ ID NO: 51 - protein, 7 aa, Artificial Sequence
  Description of Artificial Sequence Synthetic peptide
GASSRAT

SEQ ID NO: 52 - protein, 9 aa, Artificial Sequence
  Description of Artificial Sequence Synthetic peptide
QQYDSSAIT